(19) United States
(12) Patent Application Publication
(10) Pub. No.: US 2018/0030424 A1
(43) Pub. Date: Feb. 1, 2018

(54) IDURONATE-2-SULFATASE AND USE THEREOF

(71) Applicant: GREEN CROSS CORPORATION, Yongin-si (KR)

(72) Inventors: Sy LEE, Yongin-si (KR); Sung-Ick PARK, Yongin-si (KR)

(73) Assignee: GREEN CROSS CORPORATION, Yongin-si (KR)

(21) Appl. No.: 15/223,647

(22) Filed: Jul. 29, 2016

(51) Int. Cl. C12N 9/16

(52) U.S. Cl. CPC C12N 9/16; C12Y 301/06013; A61K 38/00

(57) ABSTRACT

Provided is a modified iduronate-2-sulfatase (IDS) gene constructed by inserting the nucleotide of SEQ ID NO: 2 into a wild-type IDS gene. In addition to being negatively charged, the improved IDS enzyme encoded by the modified gene exhibits a sufficient retention time in blood to target the bone, so that it is more effective for treating Hunter syndrome.

### CROSS REFERENCE TO RELATED APPLICATIONS

This is a continuation of U.S. patent application Ser. No. 13/884,806 (pending), filed May 10, 2013, which is a §371 National Stage application of PCT/KR2010/007989 filed Nov. 12, 2010 the entire disclosures of the prior applications are considered part of the disclosure of the accompanying continuation application, and are hereby incorporated by reference in their entirety.

### FIELD OF THE INVENTION

The present invention relates to an improved iduronate-2-sulfatase, and the use thereof.

### BACKGROUND OF THE INVENTION

Hunter syndrome, or mucosaccharidosis type II, is a lysosomal storage disease (LSD) in which mucopolysaccharides, also known as glycosaminoglycans (GAGs), are not broken down correctly but accumulated in a body due to deficiency of iduronate-2-sulfatase (hereinafter referred to as “IDS”). As GAG continues to accumulate throughout the cells of the body, various signs of Hunter syndrome become more visible. Physical manifestations for some people with Hunter syndrome include distinct facial features, a large head and an enlarged abdomen due to hepatomegaly or splenomegaly. Representatives among the symptoms of Hunter syndrome are deafness, valvular heart disease, obstructive airway disease and sleep apnea. Also, major joints may be affected by Hunter syndrome, leading to joint stiffness and limited motion. In some cases of Hunter syndrome, central nervous system involvement leads to developmental delays and nervous system problems.

Hunter syndrome is a serious genetic disorder that primarily affects males (X-linked recessive), with an incidence among live births of males of 1 in 162,000, and creates a great burden on the patients themselves as well as on their families.

Various trials have been carried out thus far regarding the treatment of Hunter syndrome, including bone marrow grafting, enzyme replacement, and gene therapy. While bone marrow grafting may improve most symptoms remarkably, it is difficult to find an HLA (human leukocyte antigen) match for all patients. Further, bone marrow grafting is a major surgical operation accompanied by several adverse effects, including the patient's life being put under high risk if the HLA is mismatched. Designed to administer synthetic IDS externally, the enzyme replacement therapy for Hunter syndrome has the advantage of being simple. However, enzyme replacement must be continuously carried out, which incurs a high expense. Elaprase® (Shire Pharmaceuticals Group), produced from a human cell line using recombinant DNA technology, was approved by the FDA of the U.S. as an enzyme replacement treatment for Hunter syndrome. However, this drug suffers from the drawbacks of being very expensive and having too short in vivo half-life to reach the bone, thus exerting therapeutic effects limited to the liver, the spleen, and soft tissues. As for the gene therapy for Hunter syndrome, it delivers a normal IDS gene into the body with the aid of a viral vector such as adenovirus or retrovirus, or a non-viral vector. However, gene therapy is still in the developmental and experimental stage. Therefore, there is a need for a novel therapeutic agent and method for treating Hunter syndrome.

The present inventors have endeavored to overcome the disadvantages of the conventional enzyme Elaprase® and have found that an enzyme obtained by mutating a gene which encodes a wild-type IDS is very effective for the therapy of Hunter syndrome.

### SUMMARY OF THE INVENTION

Accordingly, it is an object of the present invention to provide an improved IDS which is more effective for the therapy of Hunter syndrome, as compared to conventional, wild-type IDS.

It is another object of the present invention to provide an expression vector which comprises a gene encoding the improved IDS, a host cell comprising the same therein, and a pharmaceutical composition for treating or preventing Hunter syndrome comprising the improved IDS.

It is a further object of the present invention to provide a method for treating or preventing Hunter syndrome, using the improved IDS.

In accordance with one aspect of the present invention, there is provided a gene comprising a coding sequence of a wild-type iduronate-2-sulfate (IDS) gene, with an oligonucleotide encoding 5 to 7 negatively charged amino acids inserted into the coding sequence, and a polypeptide encoded by the modified gene.

In accordance with another aspect of the present invention, there is provided an expression vector comprising the modified IDS gene, a host cell comprising the same therein, and a pharmaceutical composition for treating or preventing Hunter syndrome comprising the polypeptide as an active ingredient.

In accordance with a further aspect of the present invention, there is provided a method for treating or preventing Hunter syndrome, which comprises administering the composition to a subject in need thereof.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic view illustrating the construction of a modified IDS gene according to the present invention, comprising a coding sequence of a wild-type IDS gene into which both an oligonucleotide of SEQ ID NO: 2 encoding 6 aspartic acids and an oligonucleotide of SEQ ID NO: 3 encoding a linker are inserted.

FIG. 2: a photograph of an agarose gel on which pcR2.1-D6-IDS was run by electrophoresis after digestion with a restriction enzyme to give an improved IDS (D6-IDS).

FIG. 3: a photograph of dot blots useful for selecting cells capable of expressing improved IDS at high yield.

FIG. 4: a photograph of Western blots of IDS expressed in CHO cells transfected with Elaprase® with various concentrations (three left lanes), and with the improved IDS of the present invention (four right lanes).

FIG. 5: a photograph of an agarose gel on which effluents from various chromatographic purification steps performed with lysates of the improved IDS-transfected CHO cells were run, together with the wild-type IDS Elaprase® (lane 7), as visualized by silver staining.

FIG. 6: a photograph of an agarose gel on which effluents from various chromatographic purification steps performed with lysates of the improved IDS-transfected CHO cells were run, together with the wild-type IDS Elaprase® (lane 7), as analyzed by Western blots.

FIG. 7: a photograph of an agarose gel on which a column D effluents of lysates of the improved IDS-transfected CHO cells was run, together with the wild-type IDS Elaprase®, as analyzed by IEF (isoelectric focusing).

FIG. 8: a graph of GAG levels in urine from mice injected with the improved IDS of the present invention, Elaprase®, and GC1111.

FIG. 9: a graph of GAG levels in liver tissues from mice injected with the improved IDS of the present invention, Elaprase®, and GC1111.

### DETAILED DESCRIPTION OF THE INVENTION

Unless specified otherwise, all of technical and scientific terms used herein have the same meanings as are generally understood to those skilled in the art to which the present invention belongs.

As used herein, the term “iduronate-2-sulfatase” or “IDS” refers to an enzyme which is involved in the degradation of heparan sulfate and dermatan sulfate, the deficiency of which causes Hunter syndrome or mucosaccharidosis type II.

The term “natural type” or “wild type,” as used in the context of the enzyme, means an enzyme obtained preferably from humans or produced from host cells using a typical method known to those skilled in the art, without any modification made thereto. In contrast, the term “improved” means an enzyme which is produced after physical, chemical, biological, or genetic modifications known to those skilled in the art have been made to the wild-type, and which exhibits improved properties compared to the wild type.

The term “coding sequence of IDS,” as used herein, refers to a nucleotide sequence which consists of a leader sequence and a mature IDS sequence, encoding an IDS enzyme with a signal peptide attached thereto.

As used herein, the term “construct” is intended to refer to a nucleic acid sequence to be inserted into an expression vector, and the term “vector” means a vehicle for gene delivery.

Hereinafter, the present invention will be described in more detail.

In accordance with an aspect thereof, the present invention provides a gene comprising an coding sequence of a wild-type iduronate-2-sulfatase (IDS) gene, with an oligonucleotide encoding 5 to 7 negatively charged amino acids inserted into the coding sequence of IDS(CDS).

The wild-type IDS gene may be a CDS fragment having the nucleotide sequence of SEQ ID NO: 1, for example, registered with GenBank accession No. NM_000202, which can be obtained from human wild-type IDS cDNA by digestion with NheI/XhoI.

Designed to endow the wild-type IDS enzyme with a negative charge, the oligonucleotide encoding negatively charged amino acids functions to target the bone as well as to increase the half-life of the enzyme, thus extending the retention time of the enzyme in blood. An oligonucleotide encoding six aspartic acids (D6) represented by the nucleotide sequence of SEQ ID NO: 2 may be exemplary. In addition, oligonucleotides encoding glutamic acids alone or in combination with aspartic acids may be employed. When combined with each other, glutamic acids and aspartic acids may be arranged randomly. In order not to alter the basic framework of the wild-type IDS enzyme, the oligonucleotide is preferably inserted between the N-terminal leader sequence and the mature sequence of the IDS coding sequence. For example, in the case of SEQ ID NO: 1, the oligonucleotide is preferably inserted between the 75^{th} and 76^{th} nucleotides.

According to one embodiment of the present invention, the modified IDS gene may further have a linker between the negatively charged amino acid-encoding oligonucleotide and the coding sequence of IDS, for example, between the oligonucleotide and the mature IDS coding sequence. The linker may be an oligonucleotide having the nucleotide sequence of SEQ ID NO: 3. Alternatively, a partially modified nucleotide sequence of SEQ ID NO: 3, for example, GCG-GAA-GCT-GAA-ACT-GGC (SEQ ID NO: 6), may be used as a linker. However, so long as it does not alter the framework of the IDS enzyme, any linker may be employed in the present invention.

In a preferred embodiment of the present invention, the modified IDS gene may have the nucleotide sequence of SEQ ID NO: 4.

Moreover, in accordance with another aspect of the present invention, there is provided an improved IDS encoded by the modified IDS gene, which may have the amino acid sequence of SEQ NO: 5.

In accordance with another aspect thereof, the present invention provides an expression vector comprising the modified IDS gene of the present invention. The expression vector may be constructed by inserting the modified IDS gene at an MCS (multiple cloning site) of a backbone plasmid. In this regard, the backbone plasmid useful in the present invention may be any of commercially available expression plasmids for mammalian cells, such as pcDNA3.1, pCI, pCMV, pHA-MEX, and pMGS, and the construction of an expression vector is widely known to those skilled in the art.

In accordance with a still another aspect, the present invention provides a host cell comprising the expression vector. The host cell can be prepared by the transfection of an expression vector comprising the modified IDS gene of the present invention into a cell using a typical method known to those skilled in the art. No particular limitations are imparted to the kind of the host cells, but human cell lines or CHO (Chinese hamster ovary) cells may be preferred.

In accordance with a further aspect, the present invention provides a pharmaceutical composition for treating or preventing Hunter syndrome comprising a polypeptide encoded by the modified IDS gene as an active ingredient. The polypeptide can be produced, as described above, from the host cell transfected with an expression vector comprising the modified IDS gene of the present invention. The production method is not specifically limited, and may be achieved by culturing the transfected cells in an appropriate culture medium for mammalian cells for such a period of time so as to allow the maximal expression of the enzyme, e.g., 10 days or longer, and purifying the polypeptide from the cell culture using a typical method, e.g., ion exchange chromatography, column chromatography, filtration, and concentration.

Meanwhile, the pharmaceutical composition according to the present invention may be provided in the form of a formulation of the polypeptide and optionally with a pharmaceutically acceptable carrier. As used herein, the term “pharmaceutically acceptable” carrier refers to a non-toxic, physiologically compatible vehicle for the active ingredient, which is suitable for ingestion by humans without undue toxicity, incompatibility, instability, irritation, allergic response, and the like.

The composition according to the present invention may be formulated with a suitable vehicle depending on the administration route taken. The formulation according to the present invention may be administered orally or parenterally, but is not limited thereto. For parenteral administration, a route selected from among transdermal, intranasal, intraperitoneal, intramuscular, subcutaneous or intravenous routes may be taken.

For oral administration, the pharmaceutical composition may be formulated in combination with a suitable oral vehicle into powders, granules, tablets, pills, troches, capsules, liquids, gels, syrups, suspensions, and wafers using a method known in the art. Examples of the suitable vehicle useful in the formulation include sugars such as lactose, dextrose, sucrose, sorbitol, mannitol, xylitol, erythritol, and maltitol, starches such as corn starch, wheat starch, rice starch, and potato starch, celluloses such as cellulose, methyl cellulose, sodium carboxymethyl cellulose, and hydroxypropyl methyl cellulose, and fillers such as gelatin and polyvinylpyrrolidone. Optionally, the formulation may further comprise a disintegrant such as crosslinked polyvinylpyrrolidone, agar, alginic acid, or sodium alginate. In addition, an anti-agglomerating agent, a lubricant, a wetting agent, a fragrant, an emulsifier, and a preservative may be further employed.

Also, the composition of the present invention may be formulated in combination with a parenteral vehicle into a parenteral dosage form such as an injectable preparation, a transdermal preparation, or an intranasal inhalation using a method well known in the art. For use in injection, the formulation must be sterilized and protected from contamination by microorganisms such as bacteria and fungi. Examples of the vehicle suitable for injection may include, but are not limited to, water, ethanol, polyol (e.g., glycerol, propylene glycol, liquid polyethylene glycol, etc.), combinations thereof, and/or a vegetable oil-containing solvent or dispersion medium. More preferably, the vehicle may be an isotonic solution such as Hank's solution, Ringer's solution, triethanol amine-containing PBS (phosphate buffered saline) or injectable sterile water, 10% ethanol, 40% propylene glycol, and 5% dextrose. In order to protect the injectable preparation from microbial contamination, it may further comprise an antibacterial and antifungal agent such as paraben, chlorobutanol, phenol, sorbic acid, thimerosal, etc. Also, the injectable preparations may further comprise, in most cases, an isotonic agent such as sugar or sodium chloride. These formulations are disclosed in a document well known in the pharmaceutical field (Remington's Pharmaceutical Science, 15^{th} Edition, 1975, Mack Publishing Company, Easton, Pa.). As concerns inhalation, the formulation according to the present invention may be delivered conveniently in the form of an aerosol spray from a compressed pack or sprayer using a suitable propellant, e.g., dichlorofluoromethane, trichlorofluoromethane, dichlorotetrafluoroethane, carbon dioxide, or a suitable gas. In case of a compressed aerosol, the unit size of a dose may be determined by a valve for delivering a metered amount. For example, capsules and cartridges of gelatin for use in an inhaler or insufflator can be formulated to contain a powder mixture of the compound and a suitable powder base such as lactose or starch for these systems.

Other suitable pharmaceutical vehicles are described in Remington's Pharmaceutical Sciences, 19^{th} ed., Mack Publishing Company, Easton, Pa., 1995.

Moreover, the formulation according to the present invention may further comprise one or more buffers (e.g., saline or PBS), carbohydrates (e.g., glucose, mannose, sucrose, or dextran), stabilizers (sodium hydrogen sulfite, sodium sulfite, or ascorbic acid), anti-oxidants, bacteriostatics, chelating agents (e.g., EDTA or glutathione), adjuvants (e.g., aluminum hydroxide), suspending agents, thickeners, and/or preservatives (benzalkonium chloride, methyl- or propyl-paraben, and chlorobutanol).

Also, the composition of the present invention may be formulated into a dosage form that allows the rapid, sustained, or delayed release of the active ingredient after being administered into mammals. An effective amount of the formulation thus prepared may be administered via a variety of routes including oral, transdermal, subcutaneous, intravenous, and intramuscular routes. The term “effective amount,” as used herein, refers to an amount of the improved IDS that allows the diagnostic or therapeutic effect to take place when administered into a patient. The dose of the formulation of the present invention may vary depending on various factors, including the route of administration, the type of subject to be treated, the type of disease to be treated, the severity of the illness, and the patient's age, gender, weight, condition, and health state. Although the content of its active ingredient varies depending on the severity of the disease to be treated, the formulation comprising the polypeptide of the present invention may be administered at an effective dose of from 10 μg to 10 mg per dosage in multiple doses a day.

In accordance with a still further aspect thereof, the present invention provides a method for treating or preventing Hunter syndrome using the improved IDS enzyme of the present invention. The method comprises administering a composition comprising the improved IDS enzyme optionally in mixture with a suitable carrier to a subject in need of treating or preventing Hunter syndrome. When administered to a patient with Hunter syndrome, the improved IDS enzyme of the present invention can make up for the deficit of IDS, thus treating, alleviating, or preventing Hunter syndrome. In addition to being negatively charged, the improved IDS enzyme of the present invention exhibits a sufficient retention time in blood to target the bone as compared to a wild type IDS, e.g., Elaprase®, so that it is more effective for treating dysostosis, a complication of Hunter syndrome.

Hereinafter, the present invention is described more specifically by the following examples, but these are provided only for illustration purposes and the present invention is not limited thereto.

### Example 1: Preparation of Modified IDS Gene

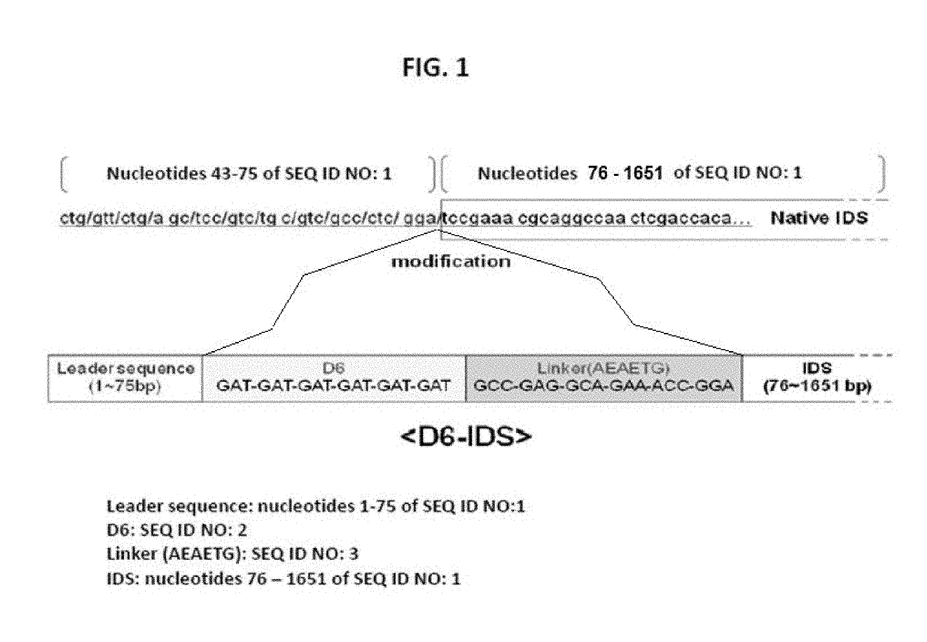

In order to solve the problem that the wild-type IDS enzyme has a short half-life in vivo and cannot reach the bone when administered, a genetic modification was made to a wild-type IDS gene. In consideration of the fact that hydroxyapatite, a main ingredient of the bone, is positively charged, an oligonucleotide encoding negatively charged amino acids was inserted into the gene so that the resulting IDS polypeptide was negatively charged. Specifically, a vector (PCR2.1-IDS) carrying a CDS fragment (˜1.7 kb, SEQ ID NO: 1) which was obtained by treating human wild-type IDS cDNA (GenBank accession No. NM_000202) with NheI/XhoI, was provided by Samsung Medical Center. An oligonucleotide consisting of six tandem codons for aspartic acid (GAT) (D6, SEQ ID NO: 2) and a linker sequence (SEQ ID NO: 3) was inserted between the 75^{th} and 76^{th} nucleotides of the CDS fragment. Because D6 and the linker were located between the N-terminal leader sequence and the IDS structural gene of the human IDS cDNA, the resulting improved IDS protein which was negatively charged was not altered in framework (see FIG. 1). The resulting recombinant vector was named pCR2.1-D6-IDS. A result of base sequencing analysis, as shown in the nucleotide sequence of SEQ ID NO: 4, confirms that D6 and the linker were inserted in order into the wild-type IDS gene represented by SEQ ID NO: 1.

### Example 2: Expression and Confirmation of Improved IDS Protein

<2-1> Construction of Modified IDS Expression Vector

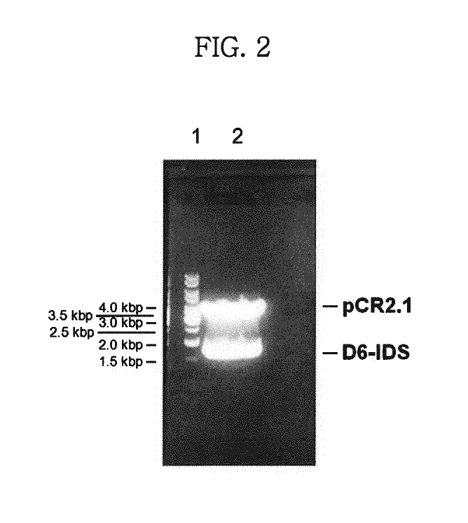

Digestion with NheI/XhoI excised a modified IDS (D6-IDS) gene from the vector prepared in Example 1 (see FIG. 2). This gene fragment was sub-cloned into a pMGS vector (Korean Patent Application No. 2000-43996; PCT/KR01/01285), which was previously treated with the same restriction enzymes, to construct a recombinant IDS expression vector pMSG-D6-IDS. The gene introduced into the vector was analyzed by base sequencing.

<2-2> Transfection of Modified IDS Expression Vector

The pMGS-D6-IDS vector constructed in <2-1> was linearized with NdeI, and purified with a QIAQuick PCR purification kit. Its DNA concentration was determined before use in transfection. CHO DG44(S)-EX cells (dhfr⁻/dhfr⁻, Colombia University) (RMCB #38) were used as host cells. The cells were maintained in a serum-free medium (glutamine-supplemented EX-CELL CD CHO medium, SAFC Bioscience) at 37±1° C. under 5±1% CO₂ with shaking at 140˜150 rpm. For passage, when the cells were grown to a concentration of 1˜2×10⁶ cells/mL, 2.5×10⁷ cells were subjected to centrifugation at 1,200 rpm for 5 min, and transferred to and suspension-cultured in another flask.

Transfection was performed on 24-well plates by electroporation. The cells in the passage culture were harvested at a concentration of 1×10⁵ cells/well, centrifuged to remove the medium, and washed once with 1×DPBS. The R buffer (included in the electroporation kit) containing the DNA (0.5 μg/well) and pDCH1P (dhfr) was added at a total volume of 12 μL/well to suspend the cells. An EX-CELL CD CHO medium supplemented with HT supplement (Invitrogen) was aliquoted in an amount of 500 μL per well, followed by applying an electric shock (1250 V, 20 msec, twice) using a 10-μL gold tip to perform transfection. Then, the cells were inoculated in a medium in each well. The cells thus transfected were incubated at 37° C. for 6 days in a 5% CO₂ incubator. When the cells were sufficiently grown, they were inoculated at a density of 1,000 cells/well into 96-well plates. The state of the cells and the formation of colonies were observed, and about 3 weeks later, 379 early adapted cell colonies were obtained.

<2-3> Selection of Modified IDS Expression Cells

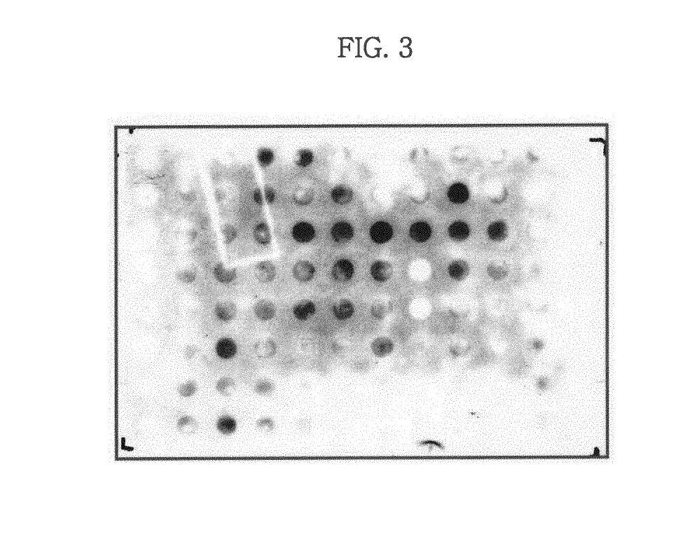

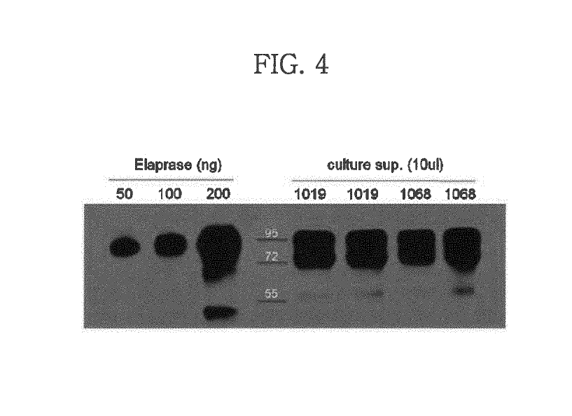

Each of the 379 cell lines was lyzed using a homogenizer, and centrifuged to give a cell lysate. Dot blotting (Protein Detector Microarray Dot Blot kit, AP Chemiluminescent, Product No. 56-12-50, KPL, USA) was performed to primarily select the top 80 cell lines which were high in IDS expression level (see FIG. 3). The selected cells were inoculated at a density of 2×10⁵ cells/well in 2 mL of the medium, and cultured for 4 days under the same conditions as described above. The cell culture was recovered and subjected to dot blotting and Western blotting (anti-human IDS antibody (R&D, AF2449)). Enzyme activity analysis (Ya. V. Voznyi et al., J. Inherit. Metab. Dis. 24 (2001) 675-680) allowed the selection of a total of 20 cell lines which expressed IDS at a high level. Western blotting on some of the selected cell lines revealed the expression of improved IDS with a size of 75˜80 kDa (see FIG. 4). The cells were cryo-preserved in vials until use.

### Example 3: Production and Purification of Improved IDS Protein

<3-1> Production of Improved IDS

One of the cryo-preserved cell lines obtained in Example 2 was rapidly thawed and placed in a germ-free centrifugation tube. After centrifugation to remove the supernatant, the resultant cell pellet was suspended in an animal component-free EX-CELL® CD CHO medium supplemented with glutamine (0.8 g/L) in a flask, and cultured at 37±1° C. in a 5±1% CO₂ atmosphere. The cells were subcultured every 2-3 days using a shaker flask until the culture volume was extended to 2 L. When the cell number was increased sufficiently to apply to a bioreactor, the cells were inoculated into the bioreactor. During the cultivation of the cells, a sample was taken from the bioreactor, and observed for cell condition under a microscope and analyzed for pH, cell concentration, cell viability, glucose concentration, glutamine concentration, and ammonia concentration. According to this information, glucose and glutamine were replenished in suitable amounts in order to prevent depletion thereof. During the cultivation, a suitable amount of TC Yeastolate (BD) was added. The cells were cultured for 10 days or longer after inoculation, and then recovered.

<3-2> Purification of Improved IDS

To effectively purify improved IDS from the cell culture, an IDS purification process was established on the basis of the IDS properties, i.e., i) a pI of IDS is 4 or less, IDS is glycosylated, and IDS has a mannose-6-phosphate.

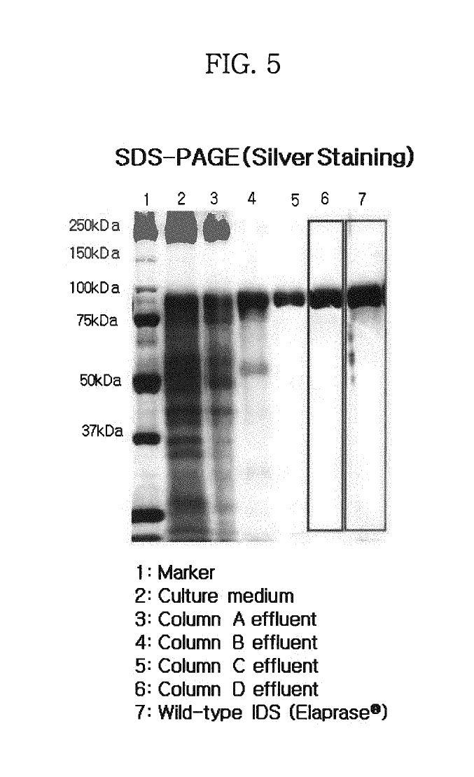

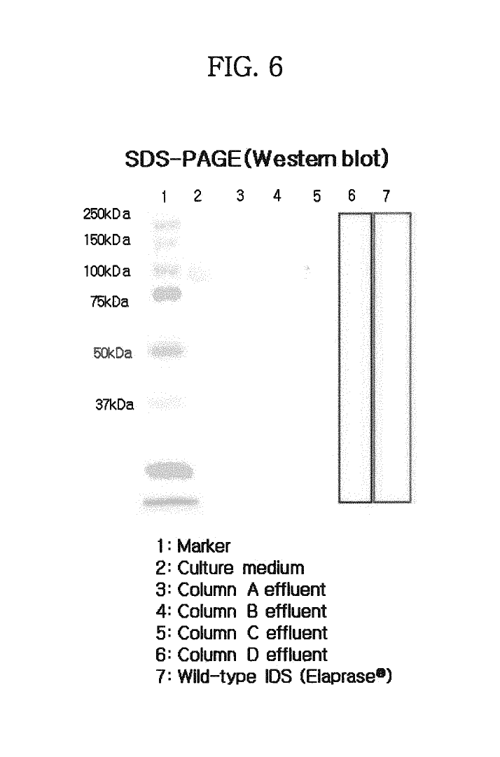

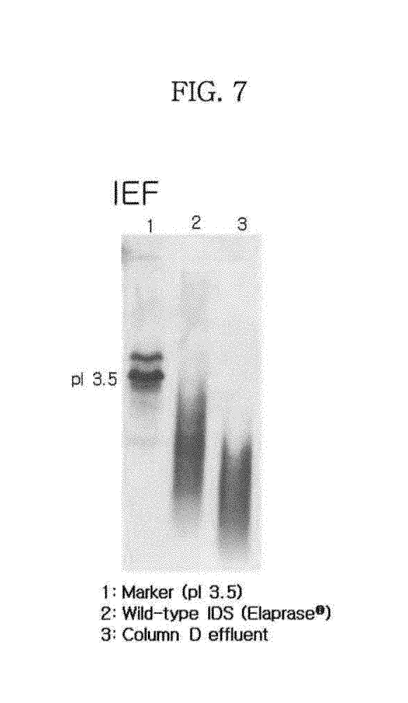

Specifically, the cell culture obtained in Example <3-1> was loaded to a column A (Anion exchange resin, GE Healthcare) equilibrated with 20 mM sodium phosphate buffer, and eluted with a buffer containing 20 mM sodium phosphate and 0.3 M sodium chloride to remove pigments and various impurities of the cell culture. Subsequently, the column A eluate was added with sodium chloride and loaded to column B (Hydrophobic Interaction resin, GE Healthcare) previously equilibrated with 20 mM sodium phosphate buffer, followed by elution with 20 mM sodium acetate to remove the pigments and impurities which remained even after the elution in column A. Thereafter, the column B eluate was loaded to column C (Cation exchange resin, GE Healthcare) previously equilibrated with 20 mM sodium phosphate buffer and eluted with 20 mM sodium acetate to remove isomers and other impurities. Finally, the column C eluate was reduced in volume by purification through 20 mM sodium acetate-equilibrated column D (Affinity resin, GE Healthcare) using 20 mM sodium phosphate as an eluting buffer. The column D eluate was concentrated using an ultrafiltration membrane (cutoff: 10,000 MWCO) to give an improved IDS protein at a concentration of 1 mg/mL or greater. The improved IDS was subjected to SDS-PAGE (silver staining and Western blotting) and IEF (isoelectric focusing), and the analysis results are shown in FIGS. 5 to 7. As can be seen in FIGS. 5 and 6, purified IDS proteins were obtained through purification steps using columns A to D in order. IEF data given in FIG. 7 indicate that the pI of the improved IDS is higher than that of the wild-tune IDS.

### Experimental Example 1: Assay for Enzyme Activity of Improved IDS Protein

The improved IDS protein was assayed for enzyme activity, in comparison with the wild-type enzymes Elaprase® (Elaprase®, Genzyme) and GC1111 (Green Cross; Otto P. van Diggelen. et. al., J. Inherit. Metab. Dis., 2001). In this regard, the IDS enzymes were reacted at various concentrations with 4MU-α-IdoA-2S (4-methylumbelliferyl-α-L-iduronide-2-sulfate.Na₂; 4-methylumbelliferone sodium salt), followed by measuring the fluorescence of the 4MU released from the substrate by the enzymes. A standard 4MU solution was used as a control and compared to the levels of 4MU released by the enzymes, improved IDS, Elsprase®, and GC1111 to determine enzyme titers.

<1-1> Preparation of Reagents for Enzyme Activity Assay
1) Sample diluent: 50 mM sodium acetate, 500 μm/mL BSA.
2) Reaction stopping solution: 0.25 M sodium carbonate/sodium bicarbonate (pH 10.7±0.2).
3) 4-MU standard solution: 100 mM 4-methylumbelliferone sodium salt.
4) Substrate diluent: 0.1 M sodium acetate/0.1 M acetic acid buffer (pH 5.0±0.2).
5) Substrate solution: solution of 5 mg of MU-αIdoA-2S (Moscerdam substrate, Netherlands) in 8.33 mL of the substrate diluent.
6) LEBT (Lysosomal Enzyme purified from Bovine Testis; Moscerdam, M2) solution: dissolved in 2.2 mL of distilled water per vial.
7) Pi/Ci buffer: 0.2 M sodium phosphate/0.1 M citric acid buffer (pH 4.5±0.2).

<1-2> Assay for Enzyme Activity

The IDS sample purified in Example 3 was serially diluted from 10 ng/mL to 5 ng/mL, to 2.5 ng/mL, and to 1.25 ng/mL using sample diluent. 20 μL of the substrate solution was added to each well of black 96-well plates while wells to be used for 4-MU standard solution remained empty. Then, 10 μL of each of the IDS sample dilutions was added to and mixed with the substrate in each well. For comparison, 10 μL of the sample diluent was also added to wells (blank). The mixtures were reacted for 4 hrs at 37° C. in an incubator while light was shielded. After the reaction, 20 μL of the Pi/Ci buffer and 10 μL of the LEBT solution were added to each well, mixed, and incubated with the reaction mixture at 37° C. for 24 hrs in an incubator in the absence of light. Afterwards, the reaction was stopped by addition of 200 μL of a reaction stopping solution to each well. Variously diluted 4-MU standard solution in the reaction stopping solution were added in an amount of 260 μL to each of the wells which were reserved to be empty. Absorbance at 355 nm/460 nm was read using a fluorescence reader (VICTOR X4, PerkinElmer). This experiment was performed in duplicate for each concentration.

The results are summarized in Table 1 below.

TABLE 1

| Sample | IDS Activity (nmol/min/μg) |
|---|---|
| Improved IDS | 56.1 |
| GC1111 | 53.8 |
| Elaprase ® | 31.8 |

As can be understood from the data of Table 1, the improved IDS according to the present invention is far superior in activity to the commercially available enzyme Elaprase®, and somewhat superior to GC1111, which is produced from CHO cells and is in a clinical trial phase.

### Experimental Example 2: Pharmacokinetic/Pharmacological Analysis of Improved IDS

Pharmacokinetic properties of the improved IDS according to the present invention were compared with those of the wild-type IDS enzymes, i.e., Elaprase®, and GC1111.

Female ICR mice (25-30 g), 6˜7 weeks old, were divided into three groups of three. Each of the three drugs was diluted to concentrations of 0.5 mg/kg, 1.0 mg/kg, and 4.5 mg/kg in saline, and 100 μL of each diluted drug was injected to the tail vein of the mice. After the mice were generally anesthetized at 5, 15, 30, 60, 120, and 180 min post-injection, whole blood (0.6 to 0.8 mL) was taken from each mouse and separated into sera and plasma. The sera were stored at −70° C. until analysis.

Serum IDS levels (ng/mL) were measured by sandwich ELISA technique using a human-specific anti-idursulfase antibody (R&D, AF2449). The experiment was performed in triplicate. Pharmacokinetic parameters were analyzed from the time-concentration curve using the nonlinear mixed effect modeling software program NONMEM (version 7, ICON Development Solutions).

Analysis results are summarized in Table 2 below.

TABLE 2

| Dose | | AUC | | |
|---|---|---|---|---|
| (mg/kg) | Drug | (μg · min/mL) | CL (mL/min/kg) | T_{1/2} (min) |
| 0.5 | Elaprase ® | 301 | 1.66 | 1249 |
| | GC1111 | 112 | 4.48 | 514 |
| | D6-IDS | 811 | 0.62 | 3234 |
| 1.5 | Elaprase ® | 417 | 3.6 | 530 |
| | GC1111 | 155 | 9.7 | 304 |
| | D6-IDS | 570 | 2.63 | 663 |
| 4.5 | Elaprase ® | 2900 | 1.55 | 1853 |
| | GC1111 | 1120 | 4.03 | 1264 |
| | D6-IDS | 2960 | 1.52 | 1875 |

AUC: Area under time-concentration curve
CL: Clearance
T_{1/2}: Clearance half-life

As can be seen in the data of Table 2, the improved IDS according to the present invention exhibited significantly excellent pharmacokinetic properties, as compared to the wild-type IDS enzymes Elaprase® and GC1111. Particularly, the improved IDS of the present invention showed the superiority of the retention time in blood for a practical clinical dose (0.5 mg/kg). These results indicate that when administered at a practical dose, the improved IDS can exert pharmaceutical efficacy in a sustained pattern as well as on a bone target.

### Experimental Example 3: Analysis of Improved IDS for Pharmaceutical Effect After Short-Term Administration

Analysis for pharmaceutical effects of the improved IDS after short-term administration was carried out by examining GAG (glycosaminoglycan) contents depending on IDS administration. In this regard, IDS-knockout mice were injected with the improved IDS, Elaprase®, and GC1111, followed by measuring the GAG levels in urine and in liver tissue.

A total of 35 B6×129 mice (8 weeks old) were divided into five groups of seven: one wild-type group (WT) and four ID-knockout groups (KO), and they were allowed to have access to water and food ad libitum. In Group 1, wild-type mice were administered 0.9% saline (100 μL), while 0.9% saline (100 μL) was administered to IDS-knockout mice in Group 2. Elaprase® 0.5 mg/kg (100 μL) was administered to IDS-knockout mice in Group 3, GC1111 0.5 mg/kg (100 μL) was administered to IDS-knockout mice in Group 4, and the improved IDS 0.5 mg/kg (100 μL) was administered to IDS-knockout mice in Group 5. The test materials were injected five times (at days 0, 7, 14, 21, and 28) to the tail vein of the mice. Urine was collected before injection and at 35 days after injection. Liver tissues were excised at 35 days after injection. Together with PBS, about 100 mg of the liver tissues was placed in a tube, homogenized using an ultrasonic homogenizer, and centrifuged. The supernatant thus formed was used for GAG analysis.

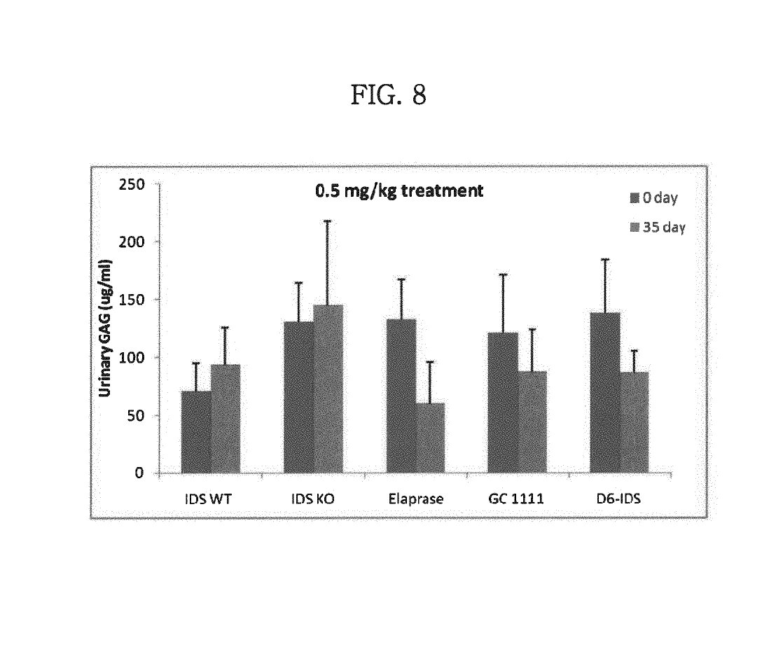

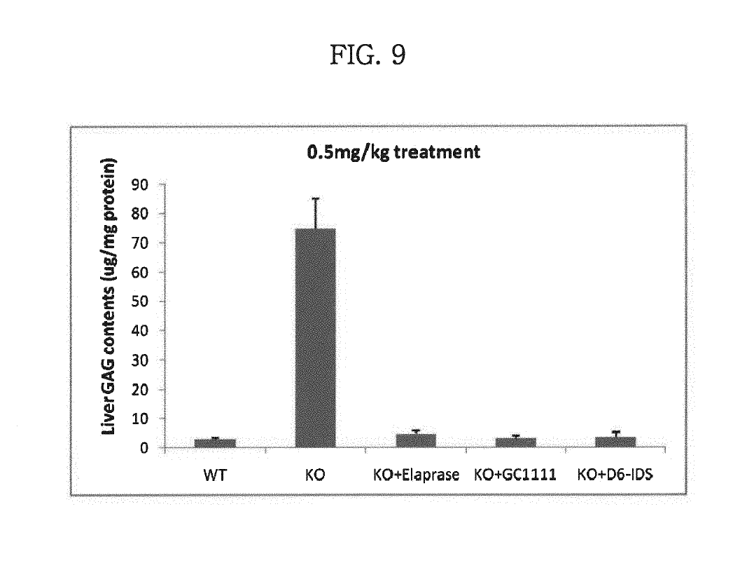

GAG levels in urine and liver tissue were measured using an sGAG assay kit (Cat. No. BP-004, KAMIYA Biochemical, USA) according to the manufacturer's instructions. The sGAG assay kit is designed to make use of a color change with the ionic bonding between anionic GAG and cationic Alcian blue dye to measure GAG contents. For liver tissues, hepatic GAG contents were normalized to the hepatic protein levels which were determined using a BCA method. Measurements of GAG contents in urine and liver tissue are shown in FIGS. 8 and 9, respectively. As can be understood from data of FIGS. 8 and 9, the improved IDS of the present invention significantly reduced GAG levels in urine and liver tissue, like the conventional drugs (Elaprase® and GC1111), as compared to the IDS-knockout mice.

Taken together, the data obtained above demonstrate that the improved IDS according to the present invention exhibits general pharmaceutical efficacy equivalent or superior to those of conventional drugs, has an extended half-life in blood, and is negatively charged, so that it can target bone tissues. Therefore, the improved IDS of the present invention can be applied to the treatment and prevention of Hunter syndrome.

While the invention has been described with respect to the above specific embodiments, it should be recognized that various modifications and changes may be made to the invention by those skilled in the art which also fall within the scope of the invention as defined by the appended claims.

What is claimed is:

1. A modified gene encoding comprising:
a nucleotide sequence encoding a wild-type iduronate-2-sulfatase (IDS) of SEQ ID NO: 1; and
an insert oligonucleotide encoding 5 to 7 negatively charged amino acids,
wherein said insert oligonucleotide is located within the coding region of the SEQ ID NO: 1 between the region encoding the N-terminal leader peptide and the region encoding the mature IDS.

2. The modified gene of claim 1, wherein the negatively charged amino acids are aspartic acid or glutamic acid.

3. The modified gene of claim 1, wherein the insert oligonucleotide consists of SEQ ID NO: 2.

4. The modified gene of claim 3, wherein the insert oligonucleotide of SEQ ID NO: 2 is inserted between 75^{th} and 76^{th} bases of the polynucleotide of SEQ ID NO: 1.

5. The modified gene of claim 1, which further comprises a linker between the insert oligonucleotide and the mature IDS-coding region.

6. The modified gene of claim 5, wherein the linker has a nucleotide sequence of SEQ ID NO: 3.

7. A modified gene consisting of:
a nucleotide sequence encoding a wild-type iduronate-2-sulfatase (IDS) of SEQ ID NO: 1; and
an insert oligonucleotide encoding 5 to 7 negatively charged amino acids,
wherein said insert oligonucleotide is located within the coding region of the gene between the region encoding the N-terminal leader peptide and the region encoding the mature IDS.

8. A polypeptide encoded by the gene of claim 1.

9. An expression vector comprising the gene of claim 1.

10. A host cell comprising the expression vector of claim 9.

11. A pharmaceutical composition for treating or prevention of Hunter syndrome, comprising the polypeptide of claim 8 as an active ingredient.

12. A method for treating or preventing Hunter syndrome, comprising administering the composition of claim 11 to a subject in need thereof.


SEQUENCE LISTING (6 sequences)

SEQ ID NO: 1 - DNA, 1653 bp, Homo sapiens
  sig_peptide (1)..(75)
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 2 - DNA, 18 bp, Artificial Sequence
  oligonucleotide sequence coding for six aspartic acids (D6)
gatgatgatgatgatgat

SEQ ID NO: 3 - DNA, 18 bp, Artificial Sequence
  linker sequence
gccgaggcagaaaccgga

SEQ ID NO: 4 - DNA, 1689 bp, Artificial Sequence
  D6-IDS sequence
atgccgccaccccggaccggccgaggccttctctggctgggtctggttctgagctccgtctgcgtcgccctcggagatgatgatgatgatgatgccgaggcagaaaccggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 5 - protein, 562 aa, Artificial Sequence
  Synthetic construct of D6-IDS amino acid sequence
MPPPRTGRGLLWLGLVLSSVCVALGDDDDDDAEAETGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 6 - DNA, 18 bp, Artificial Sequence
  linker sequence
gcggaagctgaaactggc